(19) United States
(12) Patent Application Publication
(10) Pub. No.: US 2014/0309247 A1
(43) Pub. Date: Oct. 16, 2014

(54) Systems And Methods For Detecting Scalp Follicular Inflammation In Humans

(71) Applicants: Andy Ofer Goren, Newport Beach, CA (US); John McCoy, Downey, CA (US)

(72) Inventors: Andy Ofer Goren, Newport Beach, CA (US); John McCoy, Downey, CA (US)

(21) Appl. No.: 13/863,348

(22) Filed: Apr. 15, 2013

(51) Int. Cl. C12Q 1/37; A61K 31/58; A61K 31/513

(52) U.S. Cl. CPC C12Q 1/37; A61K 31/513; A61K 31/58
USPC 514/275; 435/23; 514/284

(57) ABSTRACT

Methods, processes, systems, and apparatuses are disclosed for detecting biomarkers associated with the inflammation response in the pilosebaceous unit of the human scalp using plucked human hair and an enzyme activity assay which may for example be colorimetric or fluorometric.

### TECHNICAL FIELD

The inventions described here relate to systems and methods for detecting biomarkers associated with the inflammation response in the pilosebaceous unit of the human scalp.

### BACKGROUND

Hair loss is associated with a variety of psychological and social maladies. Prior to starting any treatment it is advantageous to predict the course, severity, and treatment options of the disease. In the field of hair loss, very few scientific diagnostic tests are currently available, and there are few methods to predict treatment response.

Moreover, the hair loss industry is populated with many products that claim to grow, improve, or replace hair. Unfortunately, only a small number of these treatments have been scientifically demonstrated to work, and of those treatments that have undergone clinical trials, many do not work equally for all patients.

Hair loss is most strongly associated with heritable, androgen-dependent pathologies. It is assumed that in genetically pre-disposed hair follicles, androgens stimulate hair follicle miniaturization, leading to a decline in hair density. However, the direct mechanism of androgen dependent hair loss has yet to be elucidated. More recently, an inflammatory process has been linked to the pathogenesis of androgenic alopecia(1).

With the addition of new scientific evidence supporting scalp inflammation in patients with androgenic alopecia(2), it would be advantageous to develop a convenient way to detect the presence and severity of scalp inflammation. A diagnostic test for scalp inflammation could be used to diagnose hair loss and track a patient's response to any number of treatments.

The pro-inflammatory cytokine cascade is associated with many biomarkers that can be detected directly in a plucked human hair. For example, pro-inflammatory cytokine synthesis is associated with the biomarkers IL-1α, IL-1β, and tumor necrosis factor (TFN). Chemokines 1L-8, MMP-8, and MMP-9 are also associated with inflammation, as well as, prostaglandins, PGHS-2, Lox, LTA4, LTB4, PLA2 and arachidonic acid generation. Recently, inflammasome activated caspase-1 was reported to be at higher concentration in the scalp of patients with androgenic alopecia (3, 4).

It would therefore be advantageous to develop systems and methods for detecting inflammation in human scalp follicles.

### BRIEF SUMMARY

The inventions described here relate to systems and methods for detecting inflammation in the scalp of patients, such as those with androgenic alopecia. This may be performed using an assay of biomarkers present in a plucked human hair. Various embodiments are possible, a number of which are exemplified here.

In one embodiment, an assay solution is described which comprises a cell lysis reagent, a buffering agent, a reducing agent for cleaving disulfide bonds of proteins, a human caspase-1 substrate comprising a marker that is catalytically cleaved upon binding to caspase-1, and glycerol.

In another embodiment, a kit is described which comprises between about 0.1 and 1 mL of an assay solution such as that described above, as well as a water-tight, transparent container with a lid that may be open and reclosed, such that after reclosure, the container maintains its water-tight property.

In another embodiment, an assay is described for detecting inflammation in the scalp of a human subject who has a scalp disorder, comprising: immersing at least one plucked hair from the subject to an assay solution such as that described above; incubating the hair within the assay solution at a temperature sufficient to promote binding between caspase-1 and said human caspase-1 substrate, for a time sufficient to allow the binding reaction to proceed essentially to completion; measuring a signal from the marker which has been cleaved upon binding to caspase-1, to derive a value representing the concentration of caspase-1; and selecting a treatment regimen that comprises the administration to the subject of an effective amount of a drug having an anti-inflammatory effect on hair follicles of the human scalp if the concentration of caspase-1 corresponds to at least 40 ng of bound caspase-1 per hair tested. When this assay is performed, and the treatment regimen is selected which includes administering a drug, another embodiment comprises a method of treating a human subject by administering an effective amount of the drug in response to that selection.

In another embodiment, a method is described for selecting, in a human subject having a scalp disorder, whether the scalp disorder is susceptible to treatment with an effective amount of a drug having an anti-inflammatory effect on scalp hair follicles, the method comprising: performing an assay such as that described above; and identifying the scalp disorder in the subject as being susceptible for treatment with an effective amount of a drug having an anti-inflammatory effect on scalp hair follicles when the concentration of caspase-1 corresponds to at least 40 ng of bound caspase-1 per hair tested.

In another embodiment, a method is described for assessing the effectiveness in a human subject of treatment with a drug having an anti-inflammatory effect on scalp hair follicles, comprising: immersing a first plucked hair, which has been plucked on a first occasion, from the subject to the assay solution such as that described above; incubating the first hair within the assay solution at a temperature sufficient to promote binding between caspase-1 and said human caspase-1 substrate, for a time sufficient to allow the binding reaction to proceed essentially to completion; measuring a first signal from the marker which has been cleaved upon binding to caspase-1, to derive a first value representing the concentration of caspase-1; immersing a second plucked hair, which has been plucked on a second occasion some period of time after the first occasion, from the subject to the assay solution of claim 1, wherein, between the first occasion and the second occasion, the subject has been administered the drug; incubating the second hair within the assay solution at a temperature sufficient to promote binding between caspase-1 and said human caspase-1 substrate, for a time sufficient to allow the binding reaction to proceed essentially to completion; measuring a second signal from the marker which has been cleaved upon binding to caspase-1, to derive a second value representing the concentration of caspase-1; and determining that the administration of the drug between the first occasion and the second occasion has been effective if the second value is less than a predetermined percentage of the first value.

### DETAILED DESCRIPTION

The description herein is provided in the context of system and method for detecting biomarkers associated with the inflammation response in the pilosebaceous unit of the human scalp. Those of ordinary skill in the art will realize that the following detailed description is illustrative only and is not intended to be in any way limiting. Other embodiments will readily suggest themselves to such skilled persons having the benefit of this disclosure. Reference will now be made in detail to implementations as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Androgenetic alopecia is extremely common, affecting approximately 60% of men and over 50% of females by the age of 60. Currently, there are two FDA approved medications for the treatment of androgenetic alopecia, finasteride and minoxidil. However, finasteride therapies that are successful at hair re-growth and maintenance in males have failed to show significant improvement in females.

In accordance with one approach described herein, a patient's hair follicle sample may be obtained. Preferably, at least two hair follicles may be obtained, so that if only one is analyzed, there will be at least one backup if needed.

In one embodiment, a method is provided for detecting inflammation in the scalp of patients with an assay of caspase-1 present in a plucked human hair, comprising the steps of: obtaining a hair follicle sample from the subject, the sample comprising at least one hair follicle; combining the hair follicle sample with an assay solution; incubating the sample for a pre-determined time, at a pre-determined temperature; measuring a value representing the concentration of caspase-1; and comparing said value with a comparison value.

The assay solution may contain but is not limited to 1) a lysis reagent, 2) a buffering agent (e.g. 2X buffer from Biozone, Cat. No. 1068-80 or 2X phosphate buffered saline), 3) a reducing agent (e.g. 20 mM 2-mercaptoethanol), 4) a caspase-1 substrate (e.g Ac-YVAD-pNA), 5) 15% glycerol. A lysis reagent refers to a reagent that will lyse cells, thus releasing the contents of those cells for analysis. Many such lysing reagents are known in the art and are routinely used by those of skill in the art. For example, CelLytic M (Sigma-Aldrich Prod. No. C2978) is a suitable commercial product that may be used.

In one embodiment, the caspase-1 substrate may be Ac-YVAD-pNA (CAS# 149321-66-3), which is a small peptide (sequence: N-Acetyl-Tyr-Val-Ala-Asp) bound to p-nitroanilide. The peptide is catalytically cleaved so that free nitroanalide is released and can be detected colorimetrically (X max 400-405 nm). In the presence of caspase-1 the assay solution will change color from a clear solution to yellow. Other chromogenic markers are known in the art, as are methods for measuring concentration of such markers based on colorimetric measurements.

Alternatively, a fluorogenic dye may be used as a marker, and the amount of the fluorogenic dye that is cleaved as a result of binding to caspase-1 may be measured by various methods known in the art for detecting the intensity of fluorescent emissions. For example, a spectrofluorometer may be used. The concentration of caspase-1 will be approximately proportional to the fluorescent emission intensity of the sample. Methods for determining concentration based on the intensity of fluorescent emission of a given fluorescent molecule are known in the art. Suitable fluorogenic markers may include AMC (7-amino-4-methylcoumarin), AFC (7-Amino-4-trifluoromethylcoumarin), or MNA (4-methoxy-2-naphthylamide).

Various chromogenic and fluorogenic caspase-1 substrates are known in the art, including without limitation Ac-YVAD-AMC (N-Acetyl-Tyr-Val-Ala-Asp-AMC), Ac-YVAD-AFC (N-Acetyl-Tyr-Val-Ala-Asp-AFC), Ac-VAD-AFC (N-Acetyl-Val-Ala-Asp-AFC), Ac-VAD-pNA (N-Acetyl-Val-Ala-Asp-pNA), Ac-WEAD-AMC (N-Acetyl-Trp-Glu-Ala-Asp-AMC), Ac-WVAD-AMC (N-Acetyl-Trp-Val-Ala-Asp-AMC), Ac-YEVD-AMC (N-Acetyl-Tyr-Glu-Val-Asp-AMC), Ac-VAD-MNA (N-Acetyl-Val-Ala-Asp-MNA), Ac-WEHD-AFC (N-Acetyl-Trp-Glu-His-Asp-AFC), Z-DEVD-pNA (benzyloxycarbonyl-Asp-Glu-Val-Asp-pNA), and Z-YVAD-AFC (benzyloxycarbonyl-Tyr-Val-Ala-Asp-AFC). Of these, Ac-YVAD-pNA, Ac-YVAD-AMC, and Ac-YVAD-AFC are most suitable for an assay. The others may be most suitable for measurement by immunoblotting. Other suitable substrates will be suggested from the above list, with various combinations of N-terminal moieties, peptide sequences, and marker.

In one embodiment this reaction may take place in a transparent container with a lid or other opening in which the hair follicle samples may be inserted. In one non-limiting example, the total amount of liquid in the assay container may be about 0.2 ml.

The reaction may be mixed and then incubated for approximately 4 to 16 hours at 37° C. depending on the number of hair follicles used in the assay. Mixing may be by any mixing means known in the art, including shaking the container. Where a shorter incubation time is required for a greater number of hair follicles. In one embodiment, an assay that uses one hair follicle may be incubated for approximately 16 hours. In another embodiment, an assay that uses two hair follicles may be incubated for approximately four hours.

It is expected that levels of caspase-1 activity per hair greater than 40 ng will be therapeutically significant. A lower threshold of 20 ng per hair, or a higher threshold of 80 ng per hair may also be considered significant. If multiple hairs are tested at a time, the total threshold will be multiplied by the number of hairs tested. In one embodiment, presence of caspase-1 higher than the threshold may indicate the existence of an inflammatory scalp disorder such as androgenetic alopecia. This may indicate that treatment with drugs such as minoxidil or finasteride, or other treatments for scalp disorders known in the art, are likely to be effective.

The effectiveness of a therapeutic regimen may be monitored by using the assay. For example, if the level of caspase-1 decreases after some period of time of treatment with the regimen, that may be an indication that the regimen is having an effect. The benefit of using such a test is that in the case of androgenetic alopecia, the typical method of measuring effectiveness is the observation of hair regrowth. However, such regrowth often takes months or years to develop. Thus, it is useful to have a method of measuring progress than takes far less time.

In one example, the assay solution may be created as follows: 20.5 μL of 2X buffer (Biozone Cat. No. 1068-80), 8 μL of glycerol, 20.5 μL of CelLytic M buffer (Sigma-Aldrich Prod. No. C2978), 1 μL of 1 M 2-mercaptoethanol (prepared, for example, by adding 70 μL of 2-mercaptoethanol to 930 μL water), and 5 μL of 10 mM YVAD-pNA. For preservation, this solution may be stored at −20° C. The relative amounts and concentrations may be varied according to the judgment of one of skill in the art, without significantly altering the assay. For example, one may use any amount of the lysing reagent which is sufficient to lyse as many cells as possible, without significantly interfering with the reaction. One may use any amount of the reducing agent for cleaving disulfide bonds of proteins which is sufficient to cleave as many disulfide bonds as possible, without significantly interfering with the reaction. The amount of the human caspase-1 substrate used should preferably be in excess of the expected amount of caspase-1 to be measured in the sample.

The above assay solution may be supplied as part of a kit which optionally includes a transparent container which in one embodiment may be large enough to contain approximately 0.2 mL of liquid. The container may also contain a lid which may be open to insert one or more hair follicles, and then reclosed for incubation and/or analysis. The same container may be used for analysis, and in one embodiment may be designed to fit within a colorimetric or fluorimetric analyzer without transferring to a separate container.

### REFERENCES

1. Mahé, Y. F., J. Michelet, N. Billoni, F. Jarrousse, B. Buan, S. Commo, D. Saint-Leger, and B. A. Bernard. Androgenetic alopecia and microinflammation. International journal of dermatology 2000: 39: 576-584.
2. Mahé, Y. F., B. Buan, N. Billoni, G. Loussouarn, J.-F. Michelet, B. Gautier, and B. A.

Bernard. Pro-inflammatory cytokine cascade in human plucked hair Skin Pharmacology and Physiology 2009: 9: 366-375.
3. Martinon, F., and J. Tschopp. Inflammatory caspases: linking an intracellular innate immune system to autoinflammatory diseases. Cell 2004: 117: 561-574.
4. Rivero Vaccari, J. P., M. E. Sawaya, F. Brand, B. P. Nusbaum, A. J. Bauman, H. M.

Bramlett, W. D. Dietrich, and R. W. Keane. Caspase-1 Level Is Higher in the Scalp in Androgenetic Alopecia. Dermatologic Surgery 2012: 38: 1033-1039.

1. An assay solution comprising:
a cell lysis reagent;
a buffering agent;
a reducing agent for cleaving disulfide bonds of proteins;
a human caspase-1 substrate comprising a marker that is catalytically cleaved upon binding to caspase-1; and
glycerol.

2. The composition of claim 1, wherein the human caspase-1 substrate is selected from the group consisting of YVAD-pNA, Ac-YVAD-AMC, Ac-YVAD-AFC.

3. The composition of claim 1, wherein the human caspase-1 substrate is selected from the group consisting of Ac-VAD-AFC, Ac-VAD-pNA, Ac-WEAD-AMC, Ac-WVAD-AMC, Ac-YEVD-AMC, Ac-VAD-MNA, Ac-WEHD-AFC, Z-DEVD-pNA, and Z-YVAD-AFC.

4. The composition of claim 1, wherein the marker is chromogenic;

5. The composition of claim 1, wherein the marker is fluorogenic;

6. A kit comprising:
a water-tight, transparent container with a lid that may be open and reclosed, such that after reclosure, the container maintains its water-tight property; and
between about 0.1 and about 1 mL of the assay solution of claim 1.

7. An assay for detecting inflammation in the scalp of a human subject who has a scalp disorder, comprising:
immersing at least one plucked hair from the subject to an assay solution comprising:
a cell lysis reagent;
a buffering agent;
a reducing agent for cleaving disulfide bonds of proteins; and
a human caspase-1 substrate comprising a marker that is catalytically cleaved upon binding to caspase-1;
incubating the hair within the assay solution at a temperature sufficient to promote binding between caspase-1 and said human caspase-1 substrate, for a time sufficient to allow the binding reaction to proceed essentially to completion;
measuring a signal from the marker which has been cleaved upon binding to caspase-1, to derive a value representing the concentration of caspase-1; and
selecting a treatment regimen that comprises the administration to the subject of an effective amount of a drug having an anti-inflammatory effect on hair follicles of the human scalp if the concentration of caspase-1 corresponds to at least 40 ng of bound caspase-1 per hair tested.

8. A method of treating a human subject with a scalp disorder, comprising:
a step of administering to the subject an effective amount of a drug having an anti-inflammatory effect on hair follicles in the human scalp, wherein the assay of claim 7 has been performed with respect to the subject, and wherein said treatment regimen comprises said step of administering.

9. The method of claim 8, wherein the scalp disorder is androgenetic alopecia, and the drug is selected from the group consisting of minoxidil and finasteride.

10. A method for selecting, in a human subject having a scalp disorder, whether the scalp disorder is susceptible to treatment with an effective amount of a drug having an anti-inflammatory effect on scalp hair follicles, the method comprising:
performing the assay of claim 7; and
identifying the scalp disorder in the subject as being susceptible for treatment with an effective amount of a drug having an anti-inflammatory effect on scalp hair follicles when the concentration of caspase-1 corresponds to at least 40 ng of bound caspase-1 per hair tested.

11. The method of claim 10, wherein the scalp disorder is androgenetic alopecia, and the drug is selected from the group consisting of minoxidil and finasteride.

12. A method for assessing the effectiveness in a human subject of treatment with a drug having an anti-inflammatory effect on scalp hair follicles, comprising:
immersing a first plucked hair, which has been plucked on a first occasion, from the subject to an assay solution comprising:
a cell lysis reagent;
a buffering agent;
a reducing agent for cleaving disulfide bonds of proteins; and
a human caspase-1 substrate comprising a marker that is catalytically cleaved upon binding to caspase-1;
incubating the first hair within the assay solution at a temperature sufficient to promote binding between caspase-1 and said human caspase-1 substrate, for a time sufficient to allow the binding reaction to proceed essentially to completion;
measuring a first signal from the marker which has been cleaved upon binding to caspase-1, to derive a first value representing the concentration of caspase-1;
immersing a second plucked hair, which has been plucked on a second occasion some period of time after the first occasion, from the subject to the assay solution 4-, wherein, between the first occasion and the second occasion, the subject has been administered the drug;
incubating the second hair within the assay solution at a temperature sufficient to promote binding between caspase-1 and said human caspase-1 substrate, for a time sufficient to allow the binding reaction to proceed essentially to completion;
measuring a second signal from the marker which has been cleaved upon binding to caspase-1, to derive a second value representing the concentration of caspase-1; and
determining that the administration of the drug between the first occasion and the second occasion has been effective if the second value is less than a predetermined percentage of the first value.

13. The method of claim 12, wherein the predetermined percentage is 100%.

14. The method of claim 12, wherein the predetermined percentage is 50%.

16. The composition of claim 7, wherein the human caspase-1 substrate is selected from the group consisting of YVAD-pNA, Ac-YVAD-AMC, Ac-YVAD-AFC.

17. The composition of claim 7, wherein the human caspase-1 substrate is selected from the group consisting of Ac-VAD-AFC, Ac-VAD-pNA, Ac-WEAD-AMC, Ac-WVAD-AMC, Ac-YEVD-AMC, Ac-VAD-MNA, Ac-WEHD-AFC, Z-DEVD-pNA, and Z-YVAD-AFC.

18. The composition of claim 7, wherein the marker is chromogenic;

19. The composition of claim 7, wherein the marker is fluorogenic;
